(19) United States
(12) Patent Application Publication
(10) Pub. No.: US 2024/0075031 A1
(43) Pub. Date: Mar. 7, 2024

(54) METHOD OF TREATING HEREDITARY HEMORRHAGIC TELANGIECTASIA USING PAZOPANIB

(71) Applicant: HHT Foundation International, Inc., Monkton, MD (US)

(72) Inventor: Dennis L. SPRECHER, Blue Bell, PA (US)

(21) Appl. No.: 18/256,304

(22) PCT Filed: Dec. 7, 2021
(86) PCT No.: PCT/US2021/062211

§ 371 Date: Jun. 7, 2023

Related U.S. Application Data
(60) Provisional application No. 63/199,116, filed on Dec. 7, 2020.

(51) Int. Cl. A61K 31/506; A61K 9/48; A61P 9/14

(52) U.S. Cl. CPC A61K 31/506; A61K 9/4825; A61K 9/4858; A61K 9/4866; A61P 9/14

(57) ABSTRACT

The present disclosure provides methods of treating a subject with hereditary hemorrhagic telangiectasia using pazopanib, wherein the method includes identifying a hemorrhagic locus, determining a therapeutically effective amount of pazopanib as a function of the hemorrhagic locus, and administering the therapeutically effective amount of pazopanib to a subject for a period of at least 6 months. In addition, treatment is targeted to vascular densities throughout the body which drive hemodynamic compromise or cosmetic disfigurement. The present disclosure provides a method of ensconcing a powder form of a therapeutically effective amount of pazopanib in a housing compartment comprising a capsule and administering the housing compartment filed with the powder form of the therapeutically effective amount of pazopanib to the subject for a period of at least 6 months. The present disclosure provides a method of administering a housing compartment comprising a capsule configured to enclose a powder form of a therapeutically effective amount of pazopanib, wherein the powder form is configured to impact a pharmacokinetic element.

### BACKGROUND OF THE DISCLOSURE

Hereditary hemorrhagic telangiectasia (HHT, Osler-Weber-Rendu disease) is an autosomal dominant rare bleeding disorder affecting 1 in 5000 persons, characterized by arteriovenous malformations (AVMs) of both large and small vessels. Causative mutations have been identified in six genes thus far, all involved in the signaling cascades of angiogenesis, and include ENG, ACVRL1, SMAD4, RASA1, GDF2, and EPHB4. Severe, recurrent epistaxis is a near universal finding and chronic gastrointestinal (GI) bleeding occurs. The majority of cases encountered in clinical practice however are due to haploinsufficiency mutations within ENG and ACVRL1. Fragile telangiectasias of the nose and gastrointestinal (GI) tract are commonly found and predispose subjects towards epistaxis and GI bleeding respectively, and engenders iron-deficiency anemia. In many subjects chronic bleeding can be severe enough to ensure dependence on packed-red-blood-cells (PRBC) transfusions to keep up with continual losses despite periodic iron infusions. This chronic hemorrhage causes significant morbidity and increases mortality in HHT, as well as significantly reduces health-related quality of life for these subjects.

Despite some considerable work on products directed towards angiogenesis for HHT therapeutics, the exact mechanistic nature of the benefit is elusive. While there are no FDA-approved treatments for HHT-associated bleeding, advances in the understanding of disease pathobiology points to the role of excess vascular endothelial growth factor (VEGF), both locally and systemically. Originally, the belief was that reduction in the driver behind angiogenesis (VEGF) would lead to slower growth of new vessels, and potentially the involution of vessels whose sustenance is derived from the mere presence of this growth factor. While we cannot discount some contribution of this type of mechanism, it does not appear to explain all of our scientific view and clinical findings. Subjects with hereditary hemorrhagic telangiectasia have increased plasma levels of vascular endothelial growth factor and transforming growth factor-beta-1. This has generated intense interest in the use of anti-angiogenic therapies to manage HHT. Systemic bevacizumab, a single-target anti-VEGF-A humanized monoclonal antibody, has been shown to improve clinical outcomes of high-output heart failure from liver vascular malformations and refractory bleeding in subjects with HHT. However, the uncertainty over effective long-term dosing, potential risk for tachyphylaxis with repeated use, requisition for intravenous administration, and deleterious side-effects at oncologic doses have triggered a search for alternative agents. Thus, there remains a need for suitable treatments for HHT.

### SUMMARY OF THE DISCLOSURE

The present disclosure provides methods for treating a subject with hereditary hemorrhagic telangiectasia using pazopanib, wherein the method includes identifying a hemorrhagic locus, determining a therapeutically effective amount of pazopanib as a function of the hemorrhagic locus, and administering the therapeutically effective amount of pazopanib to a subject for a period of at least 6 months.

The disclosure also provides a method for treating a subject with hereditary hemorrhagic telangiectasia using pazopanib, wherein the method includes ensconcing a powder form of a therapeutically effective amount of pazopanib in a housing compartment that includes a capsule and administering the housing compartment filled with the powder form of the therapeutically effective amount of pazopanib to the subject for a period of at least 6 months.

The disclosure further provides a method for treating a subject with hereditary hemorrhagic telangiectasia using pazopanib, wherein the method includes administering, for a period of at least 6 months, a housing compartment that includes a capsule configured to enclose a powder form of a therapeutically effective amount of pazopanib, wherein the powder form is configured to impact a pharmacokinetic element.

The disclosure further provides a method for treating a subject with hereditary hemorrhagic telangiectasia using pazopanib, wherein the method includes determining a first vascular density of a subject, administering a first therapeutically effective amount of pazopanib, identifying a second vascular density of the subject following a time interval, and dispensing a second therapeutically effective amount if the second vascular density is greater than the first vascular density. In some embodiments, the time interval includes a period of time elapsed between determining the first vascular density and identifying the second vascular density. In some embodiments, the time interval includes a period of time elapsed between administering the first therapeutically effective amount of pazopanib and identifying the second vascular density.

In some embodiments of any of the above methods, the therapeutically effective amount includes a range of 25-400 mg of pazopanib. In some embodiments, the therapeutically effective amount includes a range of 50-200 mg of pazopanib. In some embodiments, the therapeutically effective amount includes a range of 100-200 mg of pazopanib.

In some embodiments, the method includes identifying a severity indicator and determining the therapeutically effective amount as a function of the severity indicator.

In some embodiments, the method includes administering the therapeutically effective amount according to a dosing regimen during the period of at least 6 months.

In some embodiments, the method includes receiving at least a pharmaceutically acceptable excipient and ensconcing the at least a pharmaceutically acceptable excipient and the therapeutically effective amount of pazopanib in the housing compartment. In some embodiments the capsule comprises a gelatin capsule. In some embodiments, the capsule comprises a film coating. In some embodiments, the housing compartment is configured to be ingestible. In some embodiments, the at least an excipient comprises magnesium stearate. In some embodiments, the at least an excipient comprises microcrystalline cellulose.

In some embodiments, the method includes administering a first therapeutically effective amount of pazopanib, waiting for a predetermined period of time, and administering a second therapeutically effective amount of pazopanib as a function of the predetermined period of time. In some embodiments, the second therapeutically effective amount is greater than the first therapeutically effective amount. In some embodiments, the second therapeutically effective amount is less than the first therapeutically effective amount. In some embodiments, the predetermined period of time comprises a range of hours. In some embodiments the range of hours includes 1-24 hours. In some embodiments, the predetermined period of time includes a range of days. In some embodiments, the range of days includes 1-365 days.

In some embodiments, the method of treating a subject with hereditary hemorrhagic telangiectasia using pazopanib includes the step of determining a cardiac failure of a subject and administering a therapeutically effective amount of pazopanib as a function of the cardiac failure. In some embodiments, the method disclosed includes determining a first cardiac failure of a subject, administering a first therapeutically effective amount of pazopanib, identifying a second cardiac failure of the subject following a time interval, and dispensing a second therapeutically effective amount of pazopanib if the second cardiac failure is greater than the first cardiac failure. In some embodiments, the cardiac failure includes a comorbidity. In some embodiments, the comorbidity includes anemia. In some embodiments, the cardiac failure includes an organ lesion. In some embodiments, the organ lesion includes a liver lesion. In some embodiments, the organ lesion includes a lung lesion.

In some embodiments, the method of treating a subject with hereditary hemorrhagic telangiectasia using pazopanib, includes the step of determining a risk of hemodynamic compromise of a subject and administering a therapeutically effective amount of pazopanib as a function of the risk. In some embodiments, the risk of hemodynamic compromise includes a comorbidity. In some embodiments, the comorbidity includes anemia. In some embodiments, the risk of hemodynamic compromise includes an organ lesion.

In some embodiments, the method of treating a subject with hereditary hemorrhagic telangiectasia using pazopanib, includes the step of determining a cosmetic issue derived from a collection of dermal vascular densities of a subject, and administering a therapeutically effective amount of pazopanib as a function of this deformity. In some embodiments, the cosmetic issue comprises an organ lesion. In some embodiments, the organ lesion includes a facial lesion.

### Definitions

To facilitate the understanding of this disclosure, a number of terms are defined below. Terms defined herein have meanings as commonly understood by a person of ordinary skill in the areas relevant to the disclosure. Terms such as “a”, “an,” and “the” are not intended to refer to only a singular entity but include the general class of which a specific example may be used for illustration. The term “or” is used to mean “and/or” unless explicitly indicated to refer to alternatives only or the alternative are mutually exclusive, although the disclosure supports a definition that refers to only alternatives and “and/or.” The terms “comprising” and “including” are understood to encompass itemized components or steps whether presented by themselves or together with one or more additional components or steps. The terminology herein is used to describe specific embodiments of the disclosure, but their usage does not limit the disclosure, except as outlined in the claims.

As used herein, the term “about” is used to indicate that a value includes the standard deviation of error for the method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, any values provided in a range of values include both the upper and lower bounds and any values contained within the upper and lower bounds.

As used herein, the terms “administer” and “administering” are used to indicate the process of providing a therapeutic, pharmaceutical, housing compartment, medication, or the like thereof to a subject. For example, providing a pharmaceutical may include providing as a function of oral ingestion, intravenous, intramuscular injection, subcutaneous injection, intrathecal injection, rectal delivery, and the like thereof.

As used herein, the term “associated with” a disease, disorder, or condition refers to a relationship, either causative or correlative, between an entity and the occurrence or severity of a disease, disorder, or condition in a subject. For example, if a target is associated with a disease, disorder, or condition, the target may be the causative agent of the disease, disorder, or condition. For example, a virus may be the causative agent in a viral infection, bacteria may be the causative agent in a bacterial infection, a fungus may be the causative agent in a fungal infection, or a parasite may be the causative agent in a parasitic infection, a cancer cell may be the causative agent of a cancer, a toxin may be the causative agent of toxicity, or an allergen may the causative agent of an allergic reaction. The target associated with a disease, disorder, or condition may also or alternately be correlated with an increased likelihood of occurrence or an increase severity of a disease disorder, or condition.

As used herein, the term “causative mutation” is used to indicate a casual relationship between at least a first physiological mutation and/or abnormality and a disorder, disease, condition, and/or malady. For example, a physiological mutation occurring within one or more genes such as ENG, ACVRL1, SMAD4, RASA1, GDF2, and/or EPHB4 may comprise a casual relationship with the signaling cascades of angiogenesis.

As used herein, the term “cosmetic issue” is used to indicate one or more visible discolorations, markings, and/or blemishes located on a subject. In some embodiment, a cosmetic issue may be one or more discolorations that form because of abnormal angiogenesis. In some embodiments, a cosmetic issue may be one or more markings that form because of increased vascular density and/or increased vasculature formation. In some embodiments, a cosmetic issue may affect one or more alternative biological systems because of the increased vascular density and/or increased vasculature formation. In some embodiments, a cosmetic issue may not affect one or more alternative biological systems because of the increased vascular density and/or increased vasculature formation.

Many methodologies described herein include a step of “determining.” Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference. In some embodiments, determining involves analyzing medical inputs, such as but not limited to medications, laboratory results, images, diagnostic reports, physician notes, medical records, and the like thereof.

As used herein, the term “dosage form” refers to a physically discrete unit of an active compound (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

As used herein, the term “hemodynamic compromise” refers to an instability of cardiovascular functions and/or pressures of a subject. A hemodynamic compromise may include an instability comprising a substantial variation in systolic blood pressure of a subject. For example, and without limitation, a substantial variation in systolic blood pressure may include a variation of greater than and/or equal to 5 mmHg, 6 mmHg, 7 mmHg, 8 mmHg, 9 mmHg, 10 mmHg, 11, mmHg, 12 mmHg, 13 mmHg, 14 mmHg, 15 mmHg, 16 mmHg, 17 mmHg, 18 mmHg, 19 mmHg, 20 mmHg, and/or the like thereof. A hemodynamic compromise may include an instability comprising a substantial variation in diastolic blood pressure of a subject. For example, and without limitation, a substantial variation in diastolic blood pressure may include a variation of greater than and/or equal to 5 mmHg, 6 mmHg, 7 mmHg, 8 mmHg, 9 mmHg, 10 mmHg, 11, mmHg, 12 mmHg, 13 mmHg, 14 mmHg, 15 mmHg, 16 mmHg, 17 mmHg, 18 mmHg, 19 mmHg, 20 mmHg, and/or the like thereof. In some embodiments, a hemodynamic compromise may include an arrythmia, a heart rate variation (i.e., bradycardia, tachycardia, etc.), a murmur, and/or the like thereof. In some embodiments, a hemodynamic compromise may be a precursor to cardiac failure.

As used herein, the term “hemorrhagic locus” refers to an anatomical location of a hemorrhage and/or lesion at which blood or other bodily fluid is escaping and/or constructing additional flow paths or vasculature having a reduced resistance. A hemorrhagic locus may include an extremity, appendage, digit, limb, core, torso, trunk, and/or other anatomical location of a subject. For example, a hemorrhagic locus may include one or more anatomical locations within the cardiovascular system, integumentary system, gastrointestinal system, pulmonary system, skeletal system, muscular system, nervous system, endocrine system, lymphatic system, respiratory system, digestive system, urinary system, reproductive system, and the like. In some embodiments, a hemorrhagic locus may include the anatomical location of a nostril because of blood escaping a vessel adjacent to the mucosal membrane. In some embodiments, a hemorrhagic locus may include an anatomical location of a lung due to construction of additional vasculature that has a reduced resistance. In some embodiments, a hemorrhagic locus may include the anatomical location of a liver due to construction of additional vasculature that has a reduced resistance.

As used herein, the term “housing compartment” refers to a component that is configured to ensconce at least a material, substance, and/or other matter, wherein the component is configured to prevent the material, substance, and/or other matter from escaping the component spontaneously. The housing compartment may be configured to degrade, break, and/or expel the material, substance, and/or other matter as a function of one or more external stimuli such as, but not limited to temperature, pressure, volume, enzymes, chemicals, and the like.

As used herein, the terms “identify” or “identifies” refer to indicating, establishing, or recognizing the identity of an anatomical location. For example, blood or other bodily fluid being expelled from a nostril may identify an anatomical location of the nasal cavity or nasopharynx. As a further example, blood or other bodily fluid being expelled in the stool of a subject may identify an anatomical location of a gastrointestinal system.

As used herein, the term “treat,” or “treating,” refers to a therapeutic treatment of a disease or disorder (e.g., a genetic syndrome, an autoimmune disease, an infectious disease, a cancer, a toxicity, or an allergic reaction) in a subject. The effect of treatment can include reversing, alleviating, reducing severity of, curing, inhibiting the progression of, reducing the likelihood of recurrence of the disease or one or more symptoms or manifestations of the disease or disorder, stabilizing (i.e., not worsening) the state of the disease or disorder, and/or preventing the spread of the disease or disorder as compared to the state and/or the condition of the disease or disorder in the absence of the therapeutic treatment.

As used herein, the term “pazopanib” refers to a pharmaceutical comprising the Chemical Abstracts Service (CAS) number of 444731-52-6 and/or CAS number 635702-64-6. In some embodiments, pazopanib may be a pharmaceutical comprising a selective multi-targeted tyrosine kinase inhibitor capable of preventing, blocking, and/or inhibiting tumor growth, angiogenesis, or the like thereof. In some embodiments, pazopanib may be a pharmaceutical capable of increasing, enhancing, and/or promoting vascular density.

As used herein, the term “pharmaceutical composition” refers to an active compound, formulated together with one or more pharmaceutically acceptable carriers. In some embodiments, active compound is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients.

A “pharmacokinetic element,” as used herein, refers to an element of data representing one or more bodily affects on a pharmaceutical. For example, and without limitation, a pharmacokinetic element may represent data associated with liberation, absorption, distribution, metabolism, excretion, and/or the like thereof of a pharmaceutical composition and/or pazopanib. As a further non-limiting example, a pharmacokinetic element may represent data associated with peak plasma concentration (Cₘₐₓ), the time to reach the peak plasma concentration (Tₘₐₓ), the plasma concentration at 24 hours post-dose (C₂₄), and/or the like thereof of a pharmaceutical composition and/or pazopanib. The term “severity indicator” means a quantitative value representing a graveness of a biological condition, status, disease, symptom, disorder, or the like thereof. For example, a quantitative value may represent a high severity and/or graveness associated with epistaxis. In some embodiments, a severity indicator may be determined as a function of one or more severity tests (e.g., an epistaxis severity score “ESS”). In some embodiments, a severity indicator may be determined as a function of balancing a biological risk and the occurrence of severe treatment-emergent adverse events (TEAEs).

The term “therapeutically effective amount” means an amount that is sufficient, when administered to a population suffering from or susceptible to a disease, disorder, and/or condition in accordance with a dosing regimen, to treat, mitigate, prevent, and/or reverse the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to subjects in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

The term “treatment” (also “treat” or “treating”), in its broadest sense, refers to any administration of a substance, active compound, pharmaceutically acceptable carrier, pharmaceutically acceptable excipient, and/or the like thereof that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be administered to a subject who does not exhibit signs of the relevant disease, disorder and/or condition and/or of a subject who exhibits only early signs of the disease, disorder, and/or condition. Alternatively or additionally, in some embodiments, treatment may be administered to a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### BRIEF DESCRIPTION OF THE DRAWINGS

FIG. 1 is a flow diagram illustrating an exemplary embodiment of a pazopanib treatment pathway.

FIG. 2 is a flow diagram illustrating an exemplary embodiment of a subject selection path.

FIG. 3A is a diagrammatic representation illustrating an exemplary embodiment of a therapeutically effective amount of pazopanib effecting hemoglobin concentrations of a subject.

FIG. 3B is a diagrammatic representation illustrating an exemplary embodiment of a therapeutically effective amount of pazopanib effecting an epistaxis severity score of a subject.

FIG. 3C is a diagrammatic representation illustrating an exemplary embodiment of a therapeutically effective amount of pazopanib effecting a number of units of Red Blood Cell (RBC) transfusions received by a subject.

FIG. 3D is a diagrammatic representation illustrating an exemplary embodiment of a therapeutically effective amount of pazopanib effecting an amount of elemental iron infused in a subject.

FIG. 3E is a diagrammatic representation illustrating an exemplary embodiment of a therapeutically effective amount of pazopanib effecting ferritin concentrations of a subject.

FIG. 3F is a diagrammatic representation illustrating an exemplary embodiment of a therapeutically effective amount of pazopanib effecting transferrin saturation of a subject.

### DETAILED DESCRIPTION

The present disclosure provides a method for treating a subject with hereditary hemorrhagic telangiectasia using pazopanib, wherein the method comprises identifying a hemorrhagic locus, determining a therapeutically effective amount of pazopanib as a function of the hemorrhagic locus, and administering the therapeutically effective amount of pazopanib to a subject for a period of at least 6 months.

The disclosure also provides for methods of treating a subject with hereditary hemorrhagic telangiectasia using pazopanib, wherein the method comprises determining a first vascular density of a subject, administering a first therapeutically effective amount of pazopanib, identifying a second vascular density of the subject following a time interval, and dispensing a second therapeutically effective amount of pazopanib if the second vascular density is greater than the first vascular density. In some embodiments, the time interval comprises a period of time elapsed between determining the first vascular density and identifying the second vascular density. In an embodiment, a time interval may include any period of time such as, but not limited to, seconds, minutes, days, weeks, months, years, decades, and the like thereof. For example, and without limitation, a time interval may include 1 month, 2 months, 3, months, 4 months, 5 months, 6 months, 7 months, 8 months, 9 months, 10 months, 11 months, 12 months, or the like thereof. As a further non-limiting example, a time interval may include 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, or the like thereof. As a further non-limiting example, a time interval may include 1 year, 2 years, 3 years, 4 years, 5 years, 6 years, 7 years, 8 years, 9 years, 10 years, or the like thereof. In an embodiment, and without limitation, a time interval of 6 months may elapse between determining the first vascular density and identifying the second vascular density. In some embodiments, the time interval comprises a period of time elapsed between administering the first therapeutically effective amount of pazopanib and identifying the second vascular density. For example, a time interval of 3 months may elapse between administering a first therapeutically effective amount of pazopanib and identifying the second vascular density.

The present disclosure provides a treatment of a single-center institutional pazopanib treatment pathway for severe bleeding and transfusion-dependent anemia in subjects with HHT. In some embodiments, subjects may achieve a primary endpoint of achieving RBC transfusion independence per the Gale criteria within 12 months of treatment initiation and significant improvements in all secondary endpoints may be observed, such as but not limited to striking improvements in hemoglobin, ESS, RBC transfusions, iron infusions, iron stores, and/or invasive hemostatic procedures. In some embodiments, total freedom from RBC transfusions after pazopanib initiation may be achieved. Overall, substantial hemostasis may be achieved at a number of months following treatment with pazopanib such as but not limited to 3 months, 6 months, 9 months, 12 months, and the like thereof.

The present disclosure provides a method for treating a subject with HHT such that a subject experiences considerably less severe bleeding. For example, the method for treating HHT using pazopanib may result in a mean baseline ESS at 4.47 or a mean baseline hemoglobin of 10.1 gm/dL. In some embodiments, the method for treating HHT include dosing a subject with 50 mg, 100 mg, 150 mg, or 200 mg pazopanib for 12 weeks, wherein most improvements in both ESS and hemoglobin levels may occur.

In some embodiments the method for a subject with treating HHT includes identifying a hemorrhagic locus, wherein identifying is described above. For example, and without limitation, identifying a hemorrhagic locus may include locating a hemorrhage in one or more sections of the gastrointestinal system, such as but not limited to the buccal cavity, pharynx, esophagus, stomach, small intestine, large intestine, rectum, or anus. As a further non-limiting example, identifying a hemorrhagic locus may include locating a hemorrhage in one or more nostrils, nasal cavities, or sinus cavities.

In some embodiments, the method for treating a subject with HHT includes determining a therapeutically effective amount of pazopanib as a function of the hemorrhagic locus, wherein determining is described above. For example, and without limitation, the method may determine a therapeutically effective amount of 50 mg as a function of a hemorrhagic locus comprising a hemorrhage in the nostril, wherein a therapeutically effective amount of 100 mg may be determined as a function of a hemorrhagic locus comprising a hemorrhage in the small intestine. In some embodiments, determining the therapeutically effective amount may comprise identifying a severity indicator, wherein a severity indicator is described above. For example, and without limitation, a severity indicator may include a TEAE comprising hypertension, lymphocytopenia, fatigue, headache, dyspepsia, hypothyroidism, dysgeusia, hypophosphatemia, benign migratory glossitis, or the like thereof. In some embodiments, a TEAE may correlate to a Common Terminology Criteria for Adverse Events v 5.0 grade (CTCAE v 5.0 grade). For example, and without limitation, a CTCAE v 5.0 may correlate hypertension to a grade of 2, lymphocytopenia to a grade of 1-2, fatigue to a grade of 1, headache to a grade of 1, dyspepsia to a 1, hypothyroidism to a grade of 2, dysgeusia to a grade of 1, hypophosphatemia to a grade of 2, benign migratory glossitis to a grade of 1, or the like thereof.

As a further non-limiting example, a severity indicator may include an ESS score, wherein an ESS score is described above. For example, and without limitation, a score of 0.00-1.00 signifies, minimal or no epistaxis, 1.01-4.00 signifies mild epistaxis, 4.01-7.00 signifies moderate epistaxis, and 7.01-10.00 signifies severe epistaxis occurring over a specified time period. As In some embodiments, the method for treating a subject with HHT includes administering the therapeutically effective amount of pazopanib to a subject for a period of at least 6 months, wherein administering is described above. For example, and without limitation, the method may include administering the therapeutically effective amount of pazopanib to a subject via an oral ingestible for a period of at least 6 months. As a further non-limiting example, administering the therapeutically effective amount of pazopanib to a subject via an oral ingestible daily.

### Pazopanib Treatment Pathway

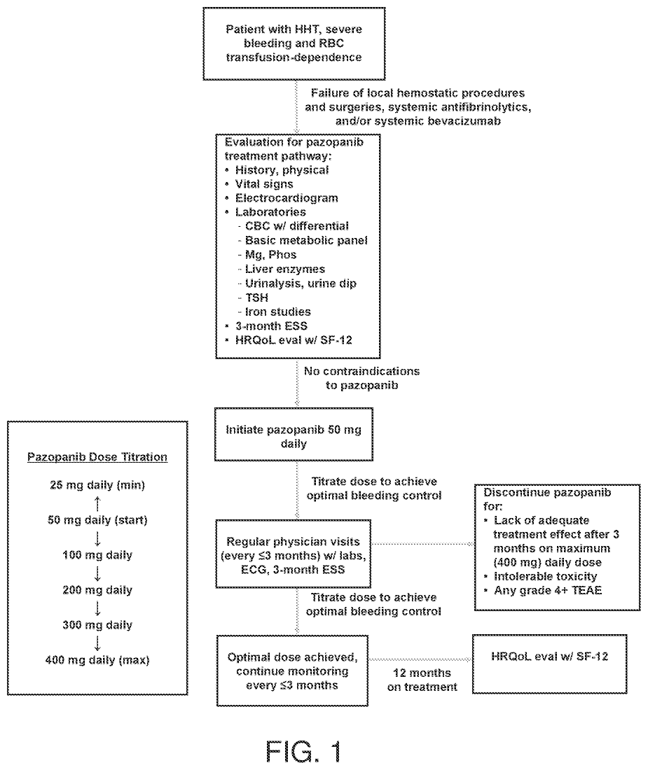

Referring now to FIG. 1, an exemplary embodiment of a pazopanib treatment pathway is illustrated. In an embodiment, subjects may be eligible for the pazopanib treatment pathway if they (1) had failed prior local hemostatic procedures and surgeries, systemic antifibrinolytics, and/or systemic bevacizumab; (2) were RBC transfusion-dependent per Gale criteria; (3) had no contraindications to pazopanib after an initial history, physical exam, and laboratory evaluation; and (4) had not received an alternative systemic anti-angiogenic agent (such as bevacizumab, thalidomide, lenalidomide, or pomalidomide), bone marrow suppressive agents, or an investigational drug within 4 weeks of treatment initiation. In some embodiments, subjects may be enrolled and administered a therapeutically effective amount of pazopanib, wherein the therapeutically effective amount may include one or more concentrations of pazopanib such as 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 52 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 g, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 152 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 g, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 252 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 g, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 352 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg.

In some embodiments, and still referring to FIG. 1, a dosing regimen may be implemented such that one or more dose escalation procedures occur, wherein a dose escalation may include increasing the therapeutically effective amount as a function of 50-100 mg increments (to a maximal dose of 400 mg daily). For example, a dose increment may include 50 mg, 100 mg, 150 mg, 200 mg, 250 mg, 300 mg, 350 mg, and the like thereof. In some embodiments, dose escalation may be performed at a predetermined period, such as but not limited to 30-day intervals. In an embodiment, and without limitation, dosing regimen may be optimized such that therapeutically effective amount of pazopanib is reduced to the lowest concentration and/or dose while still resulting in RBC transfusion independence and reducing one or more severity indicators such as an ESS. In some embodiments, dosing regimen may be implemented such that a dose reduction of therapeutically effective amount of pazopanib, wherein a does reduction may include reducing therapeutically effective amount to a minimum dose of 25 mg daily. For example, a dose reduction may include reducing therapeutically effective amount by 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 52 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, or 100 mg. Additionally or alternatively, the method may administer therapeutically effective amount of pazopanib in the morning without food due to the negative impact from concomitant administration of gastric acid-suppressing agents with pazopanib in malignancy outcomes. The method may administer therapeutically effective amount of pazopanib, wherein all proton pump inhibitors may be withdrawn prior to pazopanib administration. The method may administer therapeutically effective amount of pazopanib, wherein H2 antagonists may be prescribed to be taken once daily in the evening along with dietary discretions due to symptomatic gastroesophageal reflux disease.

In some embodiments, and still referring to FIG. 1, subjects may undergo follow-up clinical visits with vital signs, laboratory evaluations [complete blood count with white-cell differential, serum ferritin, iron and transferrin saturation, complete metabolic panel including liver function testing (transaminases, bilirubin, and alkaline phosphatase), serum phosphorous, urinalysis, and thyroid-stimulating hormone levels], electrocardiogram, and ESS calculations at a preconfigured time interval such as but not limited to 1 month, 2 months, 3 months, 4 months, 5 months, 6 months, and the like thereof per the pathway, with additional laboratory testing and visits as indicated.

### Subject Selection Path

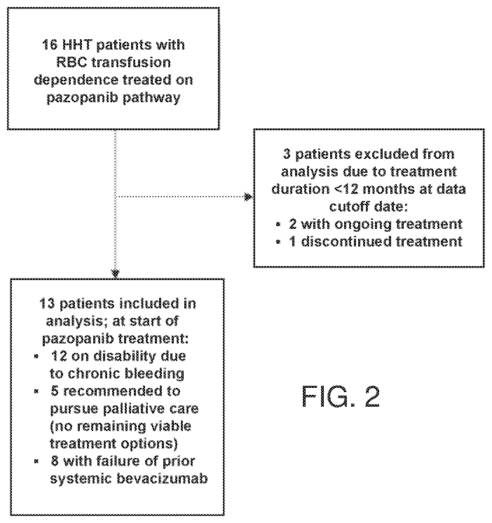

Now referring to FIG. 2, an exemplary embodiment of a subject selection path is illustrated. In some embodiments, transfusion-dependent adults with HHT may be treated with pazopanib as a function of administering oral pazopanib for epistaxis and/or gastrointestinal bleeding. For example, sixteen transfusion-dependent adults with HHT may be treated with oral pazopanib for epistaxis and/or GI bleeding; wherein thirteen may be treated for at least 12 months and may be included in the analysis; wherein the median age may be 66 (range, 53-78) years and 46% may be female. In some embodiments, subjects may suffer from one or more genetic mutations and/or causative mutations such as a mutation in at least a gene, such as but not limited to ENG, ACVRL1, or the like thereof.

### Housing Compartment

In some embodiments, the method for treating a subject with HHT includes ensconcing a powder form of a therapeutically effective amount of pazopanib in a housing compartment comprising a capsule, wherein a housing compartment is described above. As used herein a “powder form” is a form of pazopanib comprising very fine particles that may flow freely. In some embodiments, a powder form may include a dry and/or solid form of pazopanib. For example, and without limitation, the capsule may be configured to enclose 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, or the like thereof of a powdered form of pazopanib. Additionally or alternatively, a capsule may include, shell, pod, hull, tablet, and the like thereof. In some embodiments, the capsule may include a gelatin capsule such as a capsule composed of collagen, hydroxypropyl methyl cellulose, or the like thereof. In some embodiments, the capsule may include a film coating such as a thin-polymer based coating. In some embodiments the film coating may be configured to provide a protective barrier between the therapeutically effective amount of pazopanib and/or the subject's biological systems. In some embodiments, ensconcing the therapeutically effective amount of pazopanib may include ensconcing a range of 25 mg-400 mg of pazopanib. In some embodiments, ensconcing the therapeutically effective amount of pazopanib may include ensconcing a range of 50 mg-200 mg of pazopanib. In some embodiments, ensconcing the therapeutically effective amount of pazopanib may include ensconcing a range of 100 mg-200 mg of pazopanib.

In some embodiments, the method for treating a subject with HHT includes receiving at least a pharmaceutically acceptable excipient, wherein a pharmaceutically acceptable excipient is described above. For example, and without limitation, typical excipients may include: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

In some embodiments, the method for treating a subject with HHT includes ensconcing the at least a pharmaceutically acceptable excipient and the powder form of the therapeutically effective amount of pazopanib in the housing compartment comprising a capsule, wherein a housing compartment is described above. As a non-limiting example, ensconcing the at least a pharmaceutically acceptable excipient and the powder form of the therapeutically effective amount of pazopanib may include placing the at least a pharmaceutically acceptable excipient and the therapeutically effective amount of pazopanib in the capsule, shell, pod, hull, tablet, or the like thereof. In some embodiments, ensconcing the at least a pharmaceutically acceptable excipient and the powder form of the therapeutically effective amount of pazopanib may include ensconcing a range of 25 mg-400 mg of pazopanib with at least a pharmaceutically acceptable excipient. In some embodiments, ensconcing the at least a pharmaceutically acceptable excipient and the powder form of the therapeutically effective amount of pazopanib may include ensconcing a range of 50 mg-200 mg of pazopanib with at least a pharmaceutically acceptable excipient. In some embodiments, ensconcing the at least a pharmaceutically acceptable excipient and the powder form of the therapeutically effective amount of pazopanib may include ensconcing a range of 100 mg-200 mg of pazopanib with at least a pharmaceutically acceptable excipient.

In some embodiments, the housing compartment may be configured to be ingestible. For example, and without limitation, the housing compartment may be shaped in a circular, ovular, and/or rounded shape to promote ingestion. As a further non-limiting example, the housing compartment may be non-toxic and/or safe for subject consumption.

In some embodiments the method for treating a subject with hereditary hemorrhagic telangiectasia using pazopanib may include administering, for a period of at least 6 months, a housing compartment comprising a capsule configured to enclose a powder form of a therapeutically effective amount of pazopanib, wherein the powder form is configured to impact a pharmacokinetic element, wherein a pharmacokinetic element is described above. For example, and without limitation, a pharmacokinetic element of Cₘₐₓ may be impacted by increasing and/or enhancing Cₘₐₓ. For example, and without limitation, a pharmacokinetic element of Tₘₐₓ may be impacted by increasing and/or enhancing Tₘₐₓ. For example, and without limitation, a pharmacokinetic element of C₂₄ may be impacted by increasing and/or enhancing C₂₄. In some embodiments, a capsule enclosing a powder form may provide a 40% increase in exposure. In an embodiment, the powder substance may result in requiring a lower therapeutically effective amount of pazopanib, wherein the lower dose still demonstrates a therapeutic effect, in comparison to a tablet form of pazopanib. For example, and without limitation, a capsule filled with a lower quantity of pazopanib may increase the exposure and/or availability because of the power form, wherein the enhanced exposure and/or availability may provide more consistent inter-patient exposures. In some embodiments, an enhanced exposure and/or availability may provide greater homogeneous adsorption, wherein the greater homogeneous adsorption may reduce the number of subjects exhibiting data associated with pharmacokinetic outliers. For example, subjects with such extreme values could substantially add to observed toxicities.

### Vascular Density

In some embodiments, the method for treating a subject with HHT includes determining a first vascular density of a subject. As used herein, a “vascular density,” is a proportion of vessel area with blood flow over the total area measured. For example, and without limitation, vascular density may denote that a measurement area may have a 58% vascular density. In some embodiments, HHT may increase vascular density as a function of an organ lesion, wherein an organ lesion may be one or more shunts and/or flow paths of blood and/or circulatory fluids comprising a reduced resistive flow path that enhance and/or increase the amount of blood and/or circulatory fluids capable of flowing through the vasculature of the subject. In some embodiments, the method for treating a subject with HHT includes administering a first therapeutically effective amount of pazopanib, wherein the method may identify a second vascular density of the subject. In some embodiments, a first therapeutically effective amount of pazopanib may lead to microvascular rarefaction. As used in this disclosure “microvascular rarefaction,” is a process comprising a reduction of small and/or micro blood vessels that comprise the microcirculation of a subject. For example, and without limitation, pazopanib may reduce one or more vascular densities as a function of microvascular rarefaction. In an embodiment, first therapeutically effective amount of pazopanib may result in an enriched wall-to-lumen ratio in capillaries, wherein the enriched wall-to-lumen ratio may be generally observed in nail tissues, lip tissues, and/or eye tissues. In some embodiments, first therapeutically effective amount of pazopanib may lead to a blockade of endothelial nitric oxide synthase (eNOS) such that a reduction of nitric oxide (NO) may occur, wherein a reduction of NO may lead to additional platelet aggregation driving angiostatin and/or an uptick in endothelin cascade, which may lead to a shutdown and/or closure of small arterioles. In some embodiments, this cascade of events may lead to decreasing the density of vessels in arteriovenous malformations (AVM) lesions. In some embodiments, first therapeutically effective amount of pazopanib may result in a reduction of vascular density due to microvascular rarefaction. In some embodiments, the reduction of vascular density due to microvascular rarefaction may occur over a period of time, wherein a period of time includes seconds, minutes, hours, days, weeks, months, years, and the like thereof as described above.

In some embodiments, and without limitation, a vascular density may be determined as a function of one or more devices, instruments, and/or measuring tools. For example, and without limitation, an Optical Coherence Tonometry (OCT), may determine and/or identify a vascular density of a subject as a function of taking an image of vessels within about 2 mm of the skin and/or mucosal surface. In some embodiments, an OCT may be utilized to determine and/or identify one or more lesions found on a subject's nose, eye, skin, or the like thereof. In some embodiments, an OCT may be utilized to determine and/or identify one or more aspects of a hemorrhagic locus. In some embodiments, vascular density may be determined as a function of a serum concentration and/or a splay of receptor activities, wherein the serum concentration and/or the splay of receptor activities may vary and/or differ from a first subject to a second subject.

In some embodiments, and without limitation, first therapeutically effective amount of pazopanib may lead to microvascular rarefaction as a function of fully inhibiting one or more receptors. For example, and without limitation, a receptor comprising VEGFR1 and/or VEGFR2 signaling may allow for microvascular rarefaction, wherein VEGFR2 may be the primary initiator and/or promoter of angiogenesis. In some embodiments, and without limitation, the signaling from VEGFR1 may impede the signaling of VEGFR2 because the IC50 for VEGFR1 may require less pazopanib (about 0.01 umol/L), wherein VEGFR2 may require about 0.03 umol/L. In some embodiments, first therapeutically effective amount of pazopanib may promote VEGFR2 because a point may be reached such that the VEGFR1 may be fully inhibited, wherein VEGFR2 may remain in partial inhibition.

In some embodiments, the method includes dispensing a second therapeutically effective amount of pazopanib if the second vascular density is greater than the first vascular density. For example, and without limitation, the method for treating a subject with HHT may include dispensing a second therapeutically effective amount that is greater than the first therapeutically effective amount of pazopanib if the second vascular density is greater than the first vascular density. As a further non-limiting example, the method for treating a subject with HHT may include dispensing a second therapeutically effective amount that is less than the first therapeutically effective amount of pazopanib if the second vascular density is greater than the first vascular density. As a further non-limiting example, the method for treating a subject with HHT may include dispensing a second therapeutically effective amount that is equal to the first therapeutically effective amount of pazopanib if the second vascular density is greater than the first vascular density.

In some embodiments, a method for treating a subject with HHT using pazopanib may include documenting and/or storing a plurality of vascular densities associated with a subject. For example, and without limitation, documenting and/or storing a plurality of vascular densities may include determining one or more sequences of change in vascular density of a subject over a period of time, wherein a period of time includes seconds, minutes, hours, days, weeks, months, years, or the like thereof as described above. In some embodiments, and without limitation, documenting and/or storing the plurality of vascular densities may further comprise estimating one or more vascular densities as a target validation marker, wherein estimating one or more vascular densities may allow for predicting the effectiveness of the first therapeutically effective amount of pazopanib and/or the second therapeutically effective amount of pazopanib. In some embodiments, predicting the effectiveness of the first therapeutically effective amount of pazopanib and/or the second therapeutically effective amount of pazopanib may allow for an approximation of the effective tissue exposure of the first therapeutically effective amount of pazopanib and/or the second therapeutically effective amount of pazopanib such that a reduction of pharmacokinetic analysis may occur. Additionally or alternatively, in some embodiments and without limitation, the method for treating a subject with HHT may further comprise identifying one or more free molecules of pazopanib in a subject as a target validation marker. For example, and without limitation, a target validation marker may comprise identifying one or more free molecules of pazopanib that may be available to receptors of a subject as the concentration of free molecules of pazopanib may be variable depending on individual patient characteries as a function of a titration.

In some embodiments, the method for treating a subject with hereditary hemorrhagic telangiectasia using pazopanib includes determining a cardiac failure of a subject and administering a therapeutically effective amount of pazopanib as a function of the cardiac failure. The term “cardiac failure,” as used herein represents one or more reduced functions of cardiac cells and/or tissues. In an embodiment, cardiac failure may include one or more comorbidities such as but not limited to anemia, iron deficiency, and/or the like thereof. For example, and without limitation, cardiac failure may represent that a subject has anemia, wherein the anemia is leading to a reduced heart function. As a further non-limiting example, cardiac failure may denote that left ventricle is not able to maintain the oxygen demand for a subject due to a reduction in blood supply, wherein the failure to maintain the oxygen demand may result in blood volume buildup and/or backup into the lungs. In some embodiments, cardiac failure may include an organ lesion. As used in this disclosure, the term “organ lesion,” refers to one or more vascular shunts and/or flow paths that reduce a resistance of blood flow through an organ. For example, and without limitation, an organ lesion may include one or more shunts and/or flow paths of blood and/or circulatory fluids that enhance and/or increase the amount of blood and/or circulatory fluids to the cardiac cells, tissues, or the like thereof. In some embodiments, an organ lesion may include one or more shunts and/or flow paths located in the lungs, liver, kidneys, gastrointestinal tract, heart, brain, and/or the like thereof. In some embodiments, an organ lesion may include a facial lesion, wherein a “facial lesion,” as used herein, refers to a lesion located on a subject's face. In an embodiment, and without limitation, a facial lesion may be located such that the lesion is substantially visible on the subject's face. In another embodiment, the facial lesion may be located such that a discoloration and/or mark is substantially visible on the subject's face. For example, and without limitation, an organ lesion may include a lung lesion, wherein a “lung lesion,” as used herein, refers to a lesion, shunt, and/or flow path of blood and/or circulatory fluid that reduce a resistance of blood flow through one or more lung tissues. As a further non-limiting example, an organ lesion may include a liver lesion, wherein a “liver lesion,” as used herein, refers to a lesion, shunt, and/or flow path of blood and/or circulatory fluid that reduce a resistance of blood flow through one or more liver tissues.

In some embodiments, and without limitation, a cardiac failure may be determined as a function of one or more devices, instruments, and/or measuring tools. For example, and without limitation, a magnetic resonance imagining (MRI) device, may determine and/or identify a cardiac failure of a subject as a function of taking an image of organs such that lesions, shunts, flow paths with reduced resistance may be identified. In some embodiments, a computed tomography (CT) scan may be utilized to determine and/or identify one or more lesions. In some embodiments, the method for treating a subject with hereditary with HHT includes administering a therapeutically effective amount of pazopanib, wherein pazopanib is described above. In some embodiments, a therapeutically effective amount of pazopanib may enhance and/or improve one or more blood count levels, such as but not limited to red blood cell concentrations, white blood cell concentrations, hemoglobin concentrations, hematocrit concentrations, platelet concentrations, and/or the like thereof. In some embodiment, and without limitation, the therapeutically effective amount of pazopanib may reduce, treat, and/or reverse the effects of the one or more comorbidities. In some embodiments, and without limitation, the therapeutically effective amount of pazopanib may reduce, treat, and/or reverse the effects of the one or more organ lesions. For example, and without limitation, the therapeutically effective amount of pazopanib may reduce one or more shunts, lesions, and/or flow paths that reduce a resistance of blood flow through an organ.

In some embodiments, the method includes administering a first therapeutically effect amount of pazopanib and identifying a second cardiac failure of the subject following a time interval, wherein a second cardiac failure includes any of the cardiac failure as described above, and wherein a time interval includes any of the time interval as described above. In some embodiments, the method includes dispensing a second therapeutically effective amount of pazopanib if the second cardiac failure is greater than the first cardiac failure. For example, and without limitation, the method for treating a subject with HHT may include dispensing a second therapeutically effective amount of pazopanib that is greater than the first therapeutically effective amount of pazopanib if the second cardiac failure is greater than the first cardiac failure. As a further non-limiting example, the method for treating a subject with HHT may include dispensing a second therapeutically effective amount that is less than the first therapeutically effective amount of pazopanib if the second cardiac failure is greater than the first cardiac failure. As a further non-limiting example, the method for treating a subject with HHT may include dispensing a second therapeutically effective amount that is equal to the first therapeutically effective amount of pazopanib if the second cardiac failure is greater than the first cardiac failure.

### Examples

The following examples are put forth so as to provide those of ordinary skill in the art with a description of how the methods described herein may be used and evaluated, and are intended to be purely exemplary of the disclosure and are not intended to limit the scope of what the inventors regard as their disclosure.

### Effectiveness Measures of Pazopanib

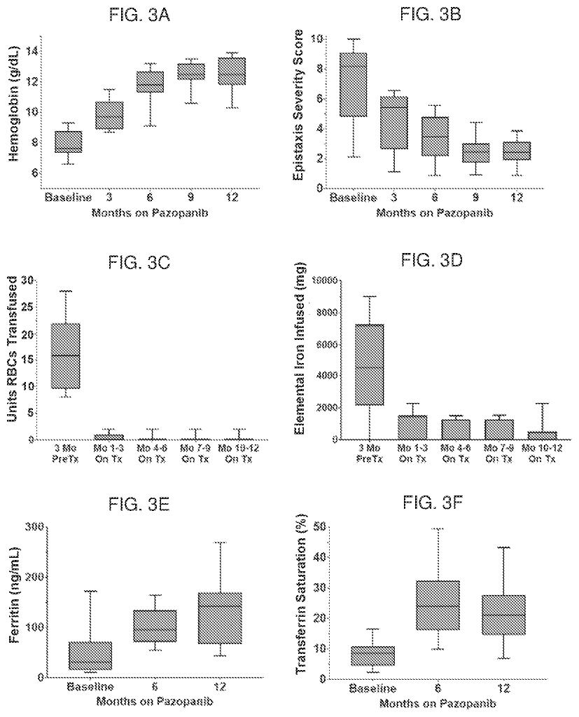

In some embodiments, this disclosure may describe pazopanib's effect on one or more endpoints such, but not limited to, a primary endpoint of achieving RBC independence per the Gale criteria during the first year of pazopanib treatment. In some embodiments, one or more secondary endpoints may include hemoglobin concentrations, epistaxis severity scores, iron infusions, baseline serum ferritin, and/or transferrin saturation. Referring now to FIG. 3, all 13 patients achieved RBC transfusion independence, with 10 patients (77%) achieving transfusion independence at 3 months of treatment and the remaining 3 patients (23%) achieving transfusion independence at 6 months of treatment. Nine patients (69%) were entirely RBC transfusion free during the 12 months following pazopanib initiation.

### Hemoglobin (Hgb)

Now referring to FIG. 3A, baseline hemoglobin at pazopanib initiation was collected and compared with hemoglobin at each time point (3, 6, 9, and 12 months) on-treatment. Baseline and on-treatment hemoglobin values were drawn at clinical nadir (just prior to a scheduled transfusion) to minimize any impact of transfusion on the value. If more than one value was measured in a given 3-month period, the average value for that period was documented for analysis.

All patients were anemic at baseline, with a mean Hgb of 7.8 (95% Cl, 7.4-8.4) g/dL. Mean hemoglobin was significantly different over the course of treatment [F(2.129, 25.55)=83.65, P<0.0001)], with increases of 2.0 (95% Cl, 1.3-2.7), 3.9 (95% Cl, 2.8-5.1), 4.6 (95% Cl, 3.4-5.7), and 4.8 (95% Cl, 3.6-5.9) g/dL at 3, 6, 9, and 12 months, respectively, relative with baseline (adjusted P<0.0001 for all pairwise comparisons). Freedom from anemia (Hgb≥11 g/dL) was observed in 15% of patients at 3 months, 85% at 6 months, 85% at 9 months, and 92% at 12 months.

### Epistaxis Severity Score (ESS)

Now referring to FIG. 3B, three-month ESS was collected by the treating physician at treatment initiation and visits at 3, 6, 9, and 12 months on-treatment. Baseline 3-month ESS at pazopanib initiation was collected and compared with 3-month ESS at each time point (3, 6, 9, and 12 months) on-treatment.

The mean ESS at baseline was 7.20 (95% Cl, 5.58-8.82) points, with 9 patients (69%) in the severe range. Mean ESS was significantly different over the course of treatment [F(1.295, 15.54)=62.43, P<0.0001)], with decreases of 2.57 (95% Cl, 1.77-3.36), 3.80 (95% Cl, 2.62-4.98), 4.76 (95% Cl, 3.10-6.42), and 4.77 (95% Cl, 3.11-6.44) points at 3, 6, 9, and 12 months, respectively, relative with baseline (adjusted P<0.0001 for all pairwise comparisons). A clinically meaningful reduction in epistaxis (ESS decrease of ≥0.71 post treatment) was observed in all patients at 3 months and maintained at 6, 9, and 12 months for all patients.

### RBC Transfusion

Now referring to FIG. 3C, a number of RBC units transfused in the 3 months of pretreatment were compared with the number of units transfused at each 3-month time interval on-treatment (1-3, 4-6, 7-9, and 10-12 months). The same analysis was performed for the total milligrams of elemental iron administered by IV infusion.

All patients were transfusion-dependent at baseline (range, 8-28 RBC units transfused in the 3 months of pretreatment). There was a significant difference in RBC units transfused over the course of treatment [χ2(4)=43.80, P<0.0001)], with a decrease in transfusions from a median (IQR) of 16.0 (9.5-22.0) RBC units in the 3 months of pretreatment to a median (IQR) of 0.0 (0.0-1.0) units at 1-3 months of on-treatment and 0.0 (0.0-0.0) at 4-6, 7-9, and 10-12 months of on-treatment (adjusted P<0.001 for all pairwise comparisons).

### Elemental Iron Transfusion

Referring now to FIG. 3D, a significant difference in milligrams of elemental iron infused over the course of treatment [χ2(4)=31.66, P<0.0001)], with a decrease in elemental iron infused from a median (IQR) of 4500 (2145-7260) milligrams in the 3 months of pretreatment to a median (IQR) of 0 (0-1500) milligrams at 1-3 months, 0 (0-1260) at 4-6 months, 0 (0-1260) at 7-9 months, and 0 (0-510) at 10-12 months of on-treatment (adjusted P<0.005 for all pairwise comparisons), FIG. 3D. Iron infusion events for each patient prior to and following pazopanib treatment are illustrated in FIG. 4. Of note, all patients were also receiving continuous oral iron supplementation (65 mg elemental iron 1-3 times daily) throughout the 3-month pretreatment period and during the 12 months on pazopanib treatment.

### Serum Ferritin

Referring now to FIG. 3E, baseline serum ferritin at pazopanib initiation was collected and compared with on-treatment values (average of values collected at 6 and 12 months). Baseline and on-treatment iron studies were drawn at nadir (prior to iron infusion or RBC transfusion, if either was given) to minimize any impact of recent iron infusion or RBC transfusion on the values.

Median (IQR) ferritin improved from 31 (16-71) ng/mL at baseline to 122 (79-154) ng/mL on-treatment (P=0.0007), FIG. 3E. Nine patients (69%) had a baseline ferritin <40 ng/dL; none had a ferritin <40 ng/dL at month 6 or 12 on-treatment.

### Serum Transferrin Saturation

Referring now to FIG. 3E, transferrin saturation at pazopanib initiation was collected and compared with on-treatment values (average of values collected at 6 and 12 months). Baseline and on-treatment iron studies were drawn at nadir (prior to iron infusion or RBC transfusion, if either was given) to minimize any impact of recent iron infusion or RBC transfusion on the values.

Median (IQR) transferrin saturation improved from 9% (5-11%) at baseline to 23% (16-31%) on-treatment (P=0.0002), FIG. 3F. No patient had a transferrin saturation ≥20% at baseline, compared with 8 patients (62%) who did at 6 months and 7 patients (54%) who did at 12 months

### Requirements for Local Hemostatic Procedures

In some embodiments, the number and type of invasive nasal and endoscopic hemostatic procedures performed to treat epistaxis and/or GI bleeding in the 12 months prior to and during the first year of pazopanib treatment were captured and compared.

The number of local hemostatic procedures decreased from a median (range) of 4 (1-5) in the 12 months of pretreatment to 0 (0-2) during the 12 months of on-treatment (P=0.0002), with 9 patients (69%) not requiring any procedures after initiating treatment. Of the 4 patients undergoing procedures, 3 underwent nasal sclerotherapy once and 1 underwent nasal sclerotherapy twice.

### Pharmacokinetics

In some embodiments, this method may result in a 20-25% increase in the Area Under the Curve (AUC) from prior publications of comparable dosing, with some modest alteration in both the peak plasma concentration (Cₘₐₓ), the time to reach the peak plasma concentration (Tₘₐₓ), and the plasma concentration at 24 hours post-dose (C₂₄) as a function of a capsule enclosing a powder substance. In some embodiments, may be similar to the results expected of a crushed tablet. No reduction in the standard error (SE), but only 6 patients may not be adequate to see this range of exposures decrease. An improvement with powder in capsules is that the variation in exposure should also be truncated based on prior crushed tablet study.

TABLE 1
Pharmacokinetics of Pazopanib

| | AUC.0-24 | Cₘₐₓ | Tₘₐₓ | C24 | |
|---|---|---|---|---|---|
| Disclosed | | | | | 6 patient PK |
| Method | | | | | study; 50 mg SD |
| Mean | 64.42 | 4.12 | 4.5/ | 2.09 | |
| | | | median | | |
| SD | 23.76 | 1.39 | | 0.89 | |
| GeoMean | 59.79 | 3.82 | | 1.90 | |
| % CV | 36.89 | 33.86 | | 42.48 | |
| Historical | | | | | Hurwitz, 2009; |
| Values | | | | | 50 mg SD |
| Mean | 51.8 | 3.3 | 3.0/ | 1.5 | |
| | | | median | | |
| % CV | 24 | 22.6 | | 45.5 | |

Dosing requires a 6-month period for full effect, such that a dose from 25 mg to 200 mg will be administered for 6-months. If the desired result is not obtained, then a dose adjustment may be appropriate after that test period to improve the outcome.

A starting dose for all patients with both epistaxis and anemia may be predetermined. However, there may be a dichotomy in the dosing based on primary source of bleed (e.g., gastrointestinal compared to nasopharynx), and the urgency of the therapeutic, (e.g., hemoglobin <11 gm/dl, vs<9.5 gm/dl requiring blood or iron supplements). Patients with substantial gastrointestinal bleeds may require between 50 and 100 mg of daily dose, while those with substantial epistaxis, based on weekly duration >25 mins, may generally require higher dosing. However, this latter group will likely be able to back down their dosing after about 6 months to a year.

In addition, those with more severe anemia (<9.5 gm/dl) may start at doses from 100-150 mg daily, while those with more moderate Hgb levels <11 mg/dl may begin at 50-100 mg. If after 6 months the clinical setting remains suboptimal, then a dose advance may be indicated.

### Other Embodiments

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims. Other embodiments are within the claims.

1. A method of treating a subject with hereditary hemorrhagic telangiectasia using pazopanib, wherein the method comprises:
identifying a hemorrhagic locus;
determining a therapeutically effective amount of pazopanib as a function of the hemorrhagic locus; and
administering the therapeutically effective amount of pazopanib to a subject for a period of at least 6 months.

2. The method of claim 1, wherein the therapeutically effective amount comprises a range of 25 mg-400 mg of pazopanib.

3. The method of claim 1, wherein the therapeutically effective amount comprises a range of 50-200 mg of pazopanib.

4. The method of claim 1, wherein the therapeutically effective amount comprises a range of 100-200 mg of pazopanib.

5. The method of claim 1, wherein determining the therapeutically effective amount further comprises identifying a severity indicator and determining the therapeutically effective amount as a function of the severity indicator.

6. The method of claim 1, wherein administering the therapeutically effective amount further comprises administering the therapeutically effective amount according to a dosing regimen during the period of at least 6 months.

7. A method of treating a subject with hereditary hemorrhagic telangiectasia using pazopanib, wherein the method comprises:
ensconcing a powder form of a therapeutically effective amount of pazopanib in a housing compartment comprising a capsule; and
administering the housing compartment filled with the powder form of the therapeutically effective amount of pazopanib to the subject for a period of at least 6 months.

8. The method of claim 7, wherein the therapeutically effective amount comprises a range of 25 mg-400 mg of pazopanib.

9. The method of claim 7, wherein the therapeutically effective amount comprises a range of 50-200 mg of pazopanib.

10. The method of claim 7, wherein the therapeutically effective amount comprises a range of 100-200 mg of pazopanib.

11. The method of claim 7, wherein ensconcing the powder form of the therapeutically effective amount of pazopanib further comprises:
receiving at least a pharmaceutically acceptable excipient; and
ensconcing the at least a pharmaceutically acceptable excipient and the powder form of the therapeutically effective amount of pazopanib in the housing compartment.

12. The method of claim 11, wherein the at least a pharmaceutically acceptable excipient comprises magnesium stearate.

13. The method of claim 11, wherein the at least a pharmaceutically acceptable excipient comprises microcrystalline cellulose.

14. The method of claim 7, wherein the capsule comprises a gelatin capsule.

15. The method of claim 7, wherein the capsule comprises a film coating.

16. The method of claim 7, wherein the housing compartment is configured to be ingestible.

17. A method of treating a subject with hereditary hemorrhagic telangiectasia using pazopanib, wherein the method comprises administering a housing compartment comprising a capsule configured to enclose a powder form of a therapeutically effective amount of pazopanib, wherein the powder form is configured to impact a pharmacokinetic element.

18. The method of claim 17, wherein the therapeutically effective amount comprises a range of 25 mg-400 mg of pazopanib.

19. The method of claim 17, wherein the therapeutically effective amount comprises a range of 50-200 mg of pazopanib.

20. The method of claim 17, wherein the therapeutically effective amount comprises a range of 100-200 mg of pazopanib.

21. The method of claim 17, wherein administering the housing compartment further comprises:
receiving at least a pharmaceutically acceptable excipient; and
ensconcing the at least a pharmaceutically acceptable excipient and the powder form of the therapeutically effective amount of pazopanib in the housing compartment.

22. The method of claim 21, wherein the at least a pharmaceutically acceptable excipient comprises magnesium stearate.

23. The method of claim 21, wherein the at least a pharmaceutically acceptable excipient comprises microcrystalline cellulose.

24. The method of claim 17, wherein the capsule comprises a gelatin capsule.

25. The method of claim 17, wherein the capsule comprises a film coating.

26. The method of claim 17, wherein the housing compartment is configured to be ingestible.

27. A method of treating a subject with hereditary hemorrhagic telangiectasia using pazopanib, wherein the method comprises:
determining a first vascular density of a subject;
administering a first therapeutically effective amount of pazopanib;
identifying a second vascular density of the subject following a time interval; and
dispensing a second therapeutically effective amount of pazopanib if the second vascular density is greater than the first vascular density.

28. The method of claim 27, wherein the therapeutically effective amount comprises a range of 25 mg-400 mg of pazopanib.

29. The method of claim 27, wherein the therapeutically effective amount comprises a range of 50-200 mg of pazopanib.

30. The method of claim 27, wherein the therapeutically effective amount comprises a range of 100-200 mg of pazopanib.

31. The method of claim 27, wherein the time interval comprises a period of time elapsed between determining the first vascular density and identifying the second vascular density.

32. The method of claim 27, wherein the time interval comprises a period of time elapsed between administering the first therapeutically effective amount of pazopanib and identifying the second vascular density.

33. A method of treating a subject with hereditary hemorrhagic telangiectasia using pazopanib, wherein the method comprises:
determining a cardiac failure of a subject; and
administering a therapeutically effective amount of pazopanib as a function of the cardiac failure.

34. The method of claim 33, wherein the therapeutically effective amount comprises a range of 25 mg-400 mg of pazopanib.

35. The method of claim 33, wherein the therapeutically effective amount comprises a range of 50-200 mg of pazopanib.

36. The method of claim 33, wherein the therapeutically effective amount comprises a range of 100-200 mg of pazopanib.

37. The method of claim 33, wherein the cardiac failure comprises a comorbidity.

38. The method of claim 33, wherein the comorbidity includes anemia.

39. The method of claim 33, wherein the cardiac failure comprises an organ lesion.

40. The method of claim 39, wherein the organ lesion includes a liver lesion.

41. The method of claim 39, wherein the organ lesion includes a lung lesion.

42. A method of treating a subject with hereditary hemorrhagic telangiectasia using pazopanib, wherein the method comprises:
determining a risk of hemodynamic compromise of a subject; and
administering a therapeutically effective amount of pazopanib as a function of the risk.

43. The method of claim 42, wherein the therapeutically effective amount comprises a range of 25 mg-400 mg of pazopanib.

44. The method of claim 42, wherein the therapeutically effective amount comprises a range of 50-200 mg of pazopanib.

45. The method of claim 42, wherein the therapeutically effective amount comprises a range of 100-200 mg of pazopanib.

46. The method of claim 42, wherein the risk of hemodynamic compromise comprises a comorbidity.

47. The method of claim 46, wherein the comorbidity includes anemia.

48. The method of claim 42, wherein the risk of hemodynamic compromise comprises an organ lesion.

49. A method of treating a subject with hereditary hemorrhagic telangiectasia using pazopanib, wherein the method comprises:
determining a cosmetic issue derived from a collection of dermal vascular densities of a subject; and
administering a therapeutically effective amount of pazopanib as a function of this deformity.

50. The method of claim 49, wherein the therapeutically effective amount comprises a range of 25 mg-400 mg of pazopanib.

51. The method of claim 49, wherein the therapeutically effective amount comprises a range of 50-200 mg of pazopanib.

52. The method of claim 49, wherein the therapeutically effective amount comprises a range of 100-200 mg of pazopanib.

53. The method of claim 49, wherein the cosmetic issue comprises an organ lesion.

54. The method of claim 53, wherein the organ lesion includes a facial lesion.
